(19) United States
(12) Patent Application Publication
(10) Pub. No.: US 2026/0253728 A1
(43) Pub. Date: Aug. 27, 2026

(54) SYSTEMS AND METHODS FOR PREDICTION OF OUTCOMES BY ANALYZING DATA

(71) Applicant: YARA GROUP, INC., San Diego, CA (US)

(72) Inventor: Farhad Sharifi, San Diego, CA (US)

(73) Assignee: YARA GROUP, INC., San Diego, CA (US)

(21) Appl. No.: 18/992,547

(22) PCT Filed: Jul. 14, 2023
(86) PCT No.: PCT/US2023/070210

§ 371 Date: Jan. 8, 2025

Related U.S. Application Data
(60) Provisional application No. 63/368,784, filed on Jul. 19, 2022.

(51) Int. Cl. G16H 50/20; G16H 10/60; G16H 20/00; G16H 50/30; G16H 50/70

(52) U.S. Cl. CPC G16H 50/20; G16H 10/60; G16H 20/00; G16H 50/30; G16H 50/70

(57) ABSTRACT

A computerized system and method for automatically estimating the likelihood of future dental health requirements, and comprises a predictive model for guiding patients to a course of treatment and facilitating preventative treatment. The system and method extracts member's health information from health administrative claims data, including clinical and pharmacy data, and estimates the probability of a dental issues. Patients are assigned a risk score and provided options for dental care associated with the risk score.

### CROSS-REFERENCE

This application claims the benefit of U.S. Provisional Application No. 63/368,784, filed Jul. 19, 2022, entitled SYSTEMS AND METHODS FOR PREDICTION OF OUTCOMES BY ANALYZING DATA which application is incorporated herein in its entirety by reference.

### NOTICE OF COPYRIGHTS

A portion of the disclosure of this patent document contains material which is subject to copyright protection. The copyright owner has no objection to the facsimile reproduction of the patent disclosure as it appears in the Patent and Trademark Office patent files or records, but otherwise reserves all copyright rights whatsoever.

### BACKGROUND

Proper dental care is an important component of comprehensive dental and important for preventing oral health related illnesses. Moreover, periodontal disease, a preventable disease that impacts 47% of adults age 30 and older, has been linked to other health problems such as cardiac complications, strokes, diabetes and respiratory problems. Unfortunately, the cost for dental care and/or access to dental insurance is known to be an impediment to receiving care.

What is needed are systems and methods for estimating future or ongoing cost of dental care.

### SUMMARY

Disclosed are systems and methods for estimating the cost of dental care. The systems and methods can result in users accessing or entering the dental care system and achieving and maintaining oral health at an earlier time.

Both the foregoing general description and the following detailed description are exemplary and explanatory only and are not restrictive of the disclosed embodiments, as claimed.

### INCORPORATION BY REFERENCE

All publications, patents, and patent applications mentioned in this specification are herein incorporated by reference to the same extent as if each individual publication, patent, or patent application was specifically and individually indicated to be incorporated by reference.
VideaHealth Dental AI Solution Receives FDA 510(k) Clearance, Establishing Industrywide Benchmark for Clinical Accuracy, Business Wire (May 2, 2022);
IN201841047063A published Jun. 19, 2020 to Artificial Learning Systems India Pvt;
IN202031055286A published Dec. 24, 2021 to Nex Fitzap Private Ltd;
IN202041038832A published Mar. 11, 2022 to Adichunachana Giri University;
IN202241009215A published Mar. 4, 2022 to Dr. A Beeula Rakajumari;
KAMINSKY, The invisible warning signs that predict your future health (Jan. 16, 2019);
RAMEZANI, Oral Cancer Screening by Artificial Intelligence Oriented Interpretation of Optical Coherence Tomography Images (2022);
RASHID, A hybrid mask RCN based tool to localize dental cavities from real-time mixed photographic images (Feb. 18, 2022);
U.S. Pat No. 10,792,004B2 issued Oct. 6, 2020 to Patel;
US2019/0340760A1 published Nov. 7, 2019 to Swank et al. ;
US2020/0146646A1 published May 14, 2020 to TUZOFF et al. ;
US2020/0388287A1 published Dec. 10, 2020 to Anushiravani et al. ;
US2021/0134440A1 published May 6, 2021 to Menavsky et al. ;
US2021/0282645A1 published Sep. 16, 2021 to Moheb;
US2021/0398275A1 published Dec. 23, 2021 to GO et al. ;
US2022/0012815A1 published Jan. 13, 2022 to Kearney et al. ;
US2022/0047160A1 published Feb. 17, 2022 to Yoo;
WO2021/240207A1 published Dec. 2, 2021 to SARABI et al. ; and
WO2021/260581A1 published Dec. 30, 2021 to GADIYAR et al.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1A is a block diagram that illustrates an exemplary environment for estimating dental outcomes and costs in accordance with the disclosure;

FIG. 1B is a block diagram that illustrates an application of the server in FIG. 1A;

FIG. 1C is block diagram illustrating development and application of the predictive model;

FIG. 1D is a table of predictive modeling techniques;

FIG. 1E is a diagram of triggers or risks factors considered relevant to the predictive model;

FIG. 2 illustrates a relationship between patient data, system data and plan data;

FIG. 3 illustrates a block diagram with an exemplary scenario for estimating dental outcomes and costs;

FIGS. 4A-B illustrate a flow diagram of a method of predicting dental outcomes and costs;

FIG. 5 illustrates variable weighting; and

FIGS. 6A-D illustrate exemplar software code.

### DETAILED DESCRIPTION

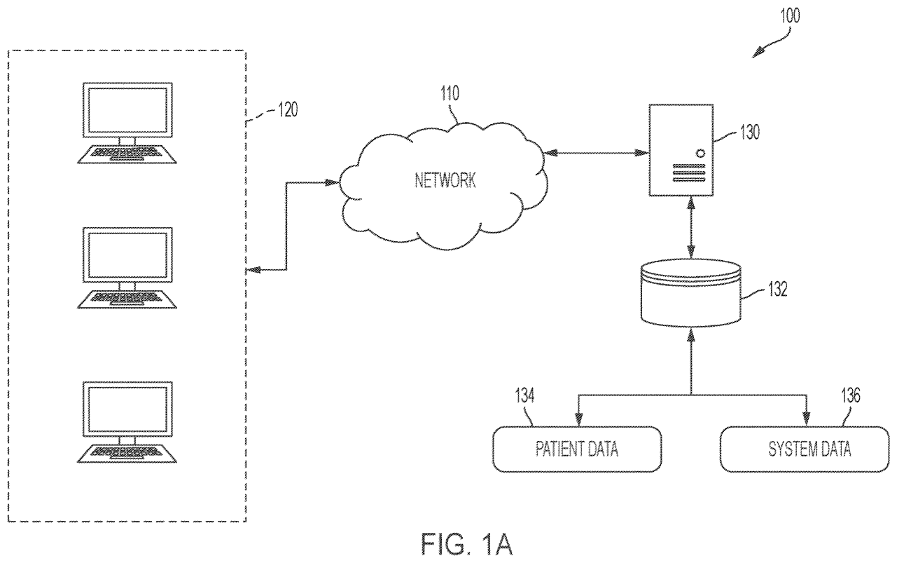

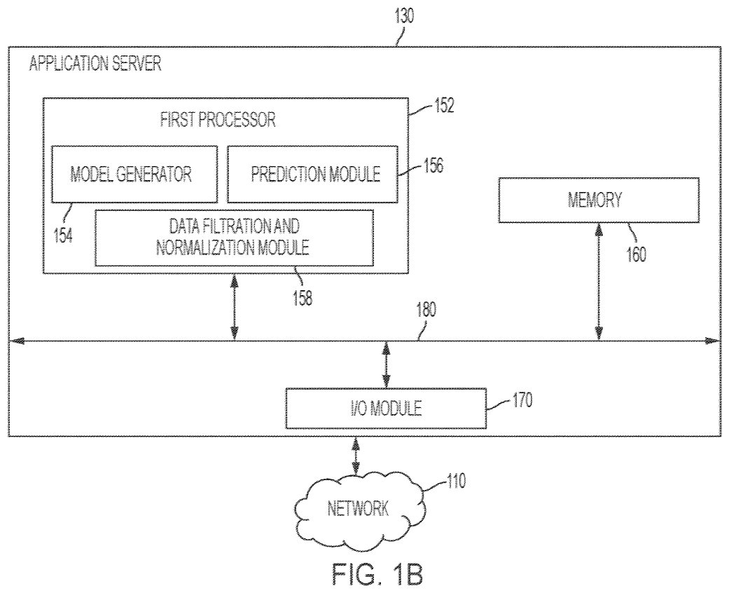

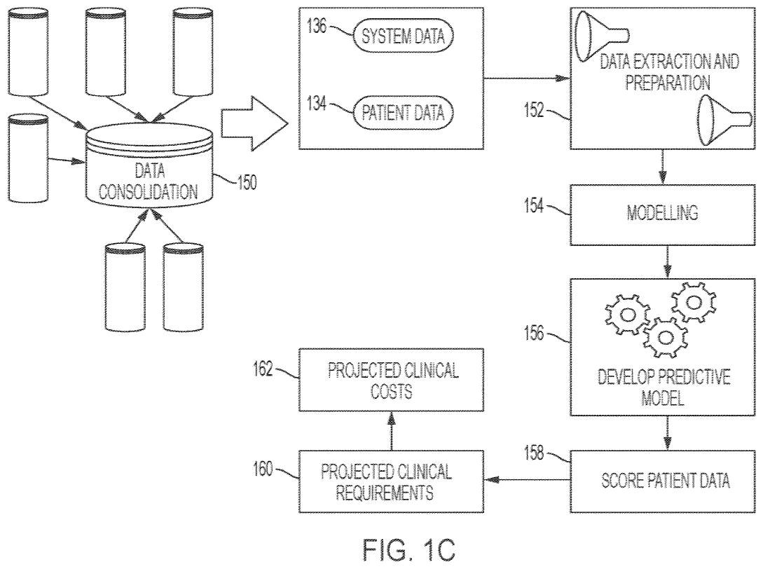

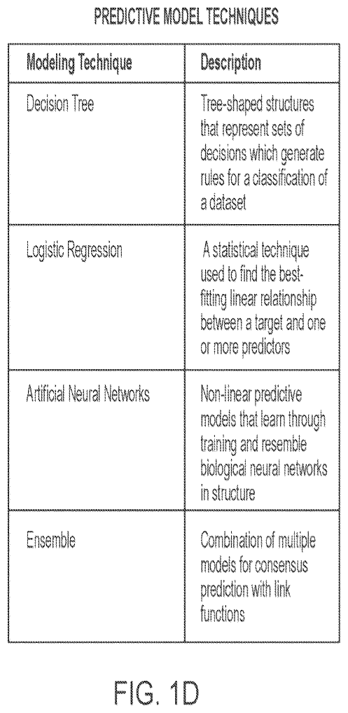

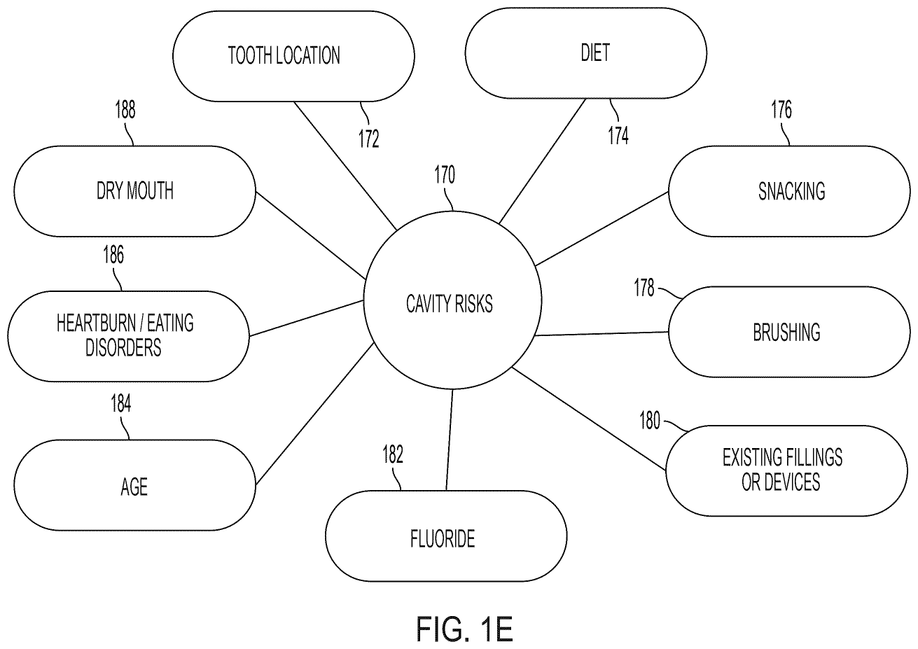

FIG. 1A is a block diagram of exemplary environment 100 for predicting dental outcomes and costs by analyzing user data and applying the user data to a model. The environment 100 includes a plurality of user devices 120, an application server 130, and a database server 132. The user devices 120, the application server 130, and the database server 132 may communicate with each other by way of a communication network 110 or any other communication means established therebetween.

Users are individuals that request a generation of a predictor for a patient after the system analyzes a patient's data using a predictor model which uses a historic dataset of other patients and a historical cost. Patient's data includes, for example, personal data (e.g., age, residence address, working address, travel history, educational level, insurance carrier, medical history, dental history, prescription medication, non-prescription medication, allergies, and financial information. Over time, ongoing medical data and dental data can be provided to the predictor model. The data of each individual patient may also include answers provided by the patient to questions. The historical data of each patient may refer to data collected based on past events pertaining to the patient. The historical data may also include data generated either manually or automatically by the patient. The historical data of the patient may further include an activity log from a biometric sensor.

The user devices 120 may include suitable logic, circuitry, interfaces, and/or code, executable by the circuitry, that may be configured to perform one or more operations for providing the data of an individual patent to the application server 130. In one exemplary scenario, the user devices 120 may refer to communication devices of the patient. The user devices 120 may be configured to allow the user to communicate with the application server 130 and the database server 132. The user devices 120 may be configured to serve as an interface for providing the patient data to the application server 130. Additionally, the user devices 120 may be configured to run or execute a software application (e.g., a mobile application or a web application), which may be hosted by the application server 130, for presenting various questions to the patient for answering. The devices 120 may be configured to communicate the answers provided by the patient to any questions provided to the patient to the application server 130. The user devices 120 may be configured to provide ongoing test and treatment data to the application server 130. Examples of the devices 120 may include, but are not limited to, mobile phones, smartphones, laptops, tablets, phablets, or other devices capable of communicating via the communication network 110.

The application server 130 may include suitable logic, circuitry, interfaces, and/or code, executable by the circuitry, that may be configured to perform one or more operations for predicting dental outcomes. The application server 130 may be a physical or cloud data processing system on which a server program runs. The application server 130 may be implemented in hardware or software, or a combination thereof. The application server 130 may be configured to host a software application which may be accessible on the internet for providing an outcome prediction service. The application server 130 may be configured to utilize the software application for retrieving the data for a patient and analyzing that data in response to a current model. The predictor models may be statistical predictive models generated by via machine learning algorithms.

After generating the predictor models, the application server 130 may be configured to utilize the predictor models during a prediction phase to predict the outcomes for a target patient based on various inputs received about the target patient (the inputs received about the target patient can be referred to as “target data”). In one example, the outcome for a target patient may include types of dental services likely to be needed, cost of associated dental services, etc. More specifically, the outcomes can include cost of projected dental services to be required over a period of time, and cost of projected dental services required over the period of time.

The application server 130 may be realized through various web-based technologies, such as, but not limited to, a Java web-framework, a . NET framework, a PHP framework, or any other web-application framework. Examples of the application server 130 include, but are not limited to, a computer, a laptop, a mini-computer, a mainframe computer, a mobile phone, a tablet, and any non-transient, and tangible machine that can execute a machine-readable code, a cloud-based server, or a network of computer systems. Various functional elements of the application server 130 have been described in detail in conjunction with FIG. 1B.

The database server 132 may include suitable logic, circuitry, interfaces, and/or code, executable by the circuitry, that may be configured to perform one or more operations for managing and storing data, such as the data of the patient, the target data of the target patient, and the predictor models. The database server 132 may be configured to receive a query from the application server 130 to extract the data stored in the database server 132. Based on the received query, the database server 132 may be configured to provide the requested data to the application server 130 over the communication network 110. In one embodiment, the database server 132 may be configured to implement as a local memory of the application server 130. In another embodiment, the database server 132 may be configured to implement as a cloud-based server. Examples of the database server 132 may include, but are not limited to, MySQL® and Oracle®.

The target patient may be an individual, whose target data may be used as input to the predictor models for predicting dental outcomes and costs. The application server 130 may be configured to obtain the target data in a manner that is similar to obtaining the test data of the patient. The application server 130 can also be configured to retrieve and/or receive the dental and bibliographic data of the target patient in real time.

The user devices 120 may include suitable logic, circuitry, interfaces, and/or code, executable by the circuitry, that may be configured to perform one or more operations for providing the target data of the target patient to the application server 130. In one exemplary scenario, the user devices 120 may refer to a communication device of the target patient. The user devices 120 may be configured to allow the target patient to communicate with the application server 130 and the database server 132. In one embodiment, the user devices 120 may be configured to provide the target data to the application server 130. For example, the user devices 120 may be configured to run or execute the software application, which is hosted by the application server 130, for presenting the questions to the target patient for answering. The user devices 120 may be configured to communicate the answers provided by the target patient to the application server 130. Examples of the user devices 120 may include, but are not limited to, mobile phones, smartphones, laptops, tablets, phablets, or other devices capable of communicating via the communication network 110.

The communication network 110 may include suitable logic, circuitry, interfaces, and/or code, executable by the circuitry, that may be configured to transmit content and messages between various entities, such as the devices 120, the application server 130, the database server 132, and/or the user devices 120. Examples of the communication network 110 may include, but are not limited to, a Wi-Fi network, a light fidelity (Li-Fi) network, a local area network (LAN), a wide area network (WAN), a metropolitan area network (MAN), a satellite network, the Internet, a fiber optic network, a coaxial cable network, an infrared (IR) network, a radio frequency (RF) network, and combinations thereof. Various entities in the environment 100 may connect to the communication network 110 in accordance with various wired and wireless communication protocols, such as Transmission Control Protocol and Internet Protocol (TCP/IP), User Datagram Protocol (UDP), Long Term Evolution (LTE) communication protocols, 5G, or any combination thereof.

In operation, the application server 130 may be configured to predict the dental outcomes in two phases, such as the learning and prediction phases. The learning phase may focus on generation of the predictor models. During the learning phase, the application server 130 may be configured to retrieve the data from the patient. The data may include the historical data of the patient, the dental and bibliographic data of the patient, and the answers provided by the patient to the questions. During the learning phase, the application server 130 may be configured to analyze the data for generating the predictor models. For example, the dental and bibliographic data corresponding to the patient may be analyzed to extract the feature values for the prediction model. The application server 130 may be further configured to utilize the analyzed test data as input for the machine learning algorithms to generate the predictor models. The analyzed test data and the predictor models may be stored in the database server 132.

A model learning phase may be followed by the prediction phase. During the prediction phase, the application server 130 may be configured to retrieve the target data of the target patient. The target data may include one or more dental and bibliographic data corresponding to the target patient, answers provided by the target patient to the questions, and/or the historical data of the target patient. The application server 130 may be further configured to analyze the target data for predicting the dental outcomes. For example, the answers provided by the target patient and the dental and bibliographic data of the target patient may be analyzed.

FIG. 1B is a block diagram that illustrates the application server 130, in accordance with an embodiment of the disclosure. The application server 130 may include a first processor 152, a memory 160, and an input/output (I/O) module 170. The first processor 152, the memory 160, and the I/O module 170 may communicate with each other by means of a communication bus 180.

The first processor 152 may include suitable logic, circuitry, interfaces, and/or code, executable by the circuitry, that may be configured to perform one or more operations for implementing the learning and prediction phases. The first processor 152 may be configured to obtain the data of the patient and the target data of the target patient. The first processor 152 may be configured to analyze the answers provided by the patient and the historic patient database. The first processor 152 may include multiple functional blocks, such as: a model generator 154, a data filtration and normalization module 158, and a prediction module 156. Examples of the first processor 152 may include, but are not limited to, an application-specific integrated circuit (ASIC) processor, a reduced instruction set computing (RISC) processor, a complex instruction set computing (CISC) processor, a field-programmable gate array (FPGA), and the like.

As will be appreciated by those skilled in the art, some parts of the patient data may be provided by the patient or extracted from the past health records (medical or dental) through any dental entity in the form of any manual or automatic channel with any types of technology. One or more parts of patient data may or may not be generated or calculated by the server itself with any possible artificial intelligence method. One or more parts of patient data may or may not be generated or calculated by the server itself with any possible non-artificial intelligence method. Some parts of patient data may or may not be received from integration with one or multiple third-party applications. Patient data may or may not include a plan or history of prior plans.

The model generator 154 and the filtration and normalization module 158 may include suitable logic, circuitry, interfaces, and/or code, executable by the circuitry, that may be configured to implement the learning phase for generating the predictor models. During the learning phase, the data may be received and analyzed. For example, the model generator 154 may be configured to analyze answers provided by the patient, the data filtration and normalization module 158 may be configured to analyze the historical data of the patient. The model generator 154 may be configured to use the normalized and filtered historical data, and the derived projected outcome for generating the predictor models. For the generation of the predictor models, the model generator 154 may be configured to use various machine learning algorithms such as, but not limited to, regression based predictive learning and neural networks based predictive leaning. In one embodiment, the model generator 154 may be further configured to update the predictor models to improve its prediction accuracy based on a feedback provided by the target patient on relevance of the predicted dental outcomes.

The data filtration and normalization module 158 may be configured to normalize and filter the historical data of the patient and the target patient. For example, the data filtration and normalization module 158 may be configured to filter the commonly used words (such as “the”, “is”, “at”, “which”, and the like) as irrelevant information from the historical data and normalize the remaining historical data to make it more meaningful. In another example, the historical data may be filtered to parse specific keywords such as, but not limited to, identifying a stream of numbers that may represent a mobile number and extracting keywords related to the patient.

The prediction module 156 may include suitable logic, circuitry, interfaces, and/or code, executable by the circuitry, that may be configured to implement the prediction phase for predicting the dental outcomes by using the target data as input to the predictor models. In one embodiment, the prediction module 156 may be configured to use the predictor models to predict dental outcomes and costs based on the analyzed historical data.

The memory 160 may include suitable logic, circuitry, interfaces, and/or code, executable by the circuitry, that may be configured to store the instructions and/or code that enable the first processor 152 to execute their operations. In one embodiment, the memory 160 may be configured to store the data. Examples of the memory 160 may include, but are not limited to, a random-access memory (RAM), a read-only memory (ROM), a removable storage drive, a hard disk drive (HDD), a flash memory, a Solid-State Drive (SSD), and the like. It will be apparent to a person skilled in the art that the scope of the disclosure is not limited to realizing the memory 160 in the application server 130, as described herein. In another embodiment, the memory 160 may be realized in form of a cloud storage working in conjunction with the application server 130, without departing from the scope of the disclosure.

The I/O module 170 may include suitable logic, circuitry, interfaces, and/or code, executable by the circuitry, that may be configured to transmit and receive data to (or form) various entities, such as the devices 120, the user devices 120, and/or the database server 132 over the communication network 110. Examples of the I/O module 170 may include, but are not limited to, an antenna, a radio frequency transceiver, a wireless transceiver, a Bluetooth transceiver, an Ethernet port, a universal serial bus (USB) port, or any other device configured to transmit and receive data. The I/O module 170may be configured to communicate with the devices 120, the user devices 120, and the database server 132 using various wired and wireless communication protocols, such as TCP/IP, UDP, LTE, 5G communication protocols, or any combination thereof.

FIG. 1C is block diagram illustrating development and application of the predictive model. In FIG. 1C, data sources and the elements that may contribute to the patient's tooth decay arc consolidated 150. Data sources can include, for example embodiment include membership information, demographic and geographic information, clinical and medical claims data, and pharmacy claims data. Persons of skill in the art of predictive modeling will appreciate that these data sources merely represent an example of the many data sources that can be used for predictive modeling. Predictors used as inputs for the predictive model such as age, gender, race and residence location from a member profile, clinical diagnosis, previous dental claims or history extracted 152 from these data sources. The disclosed system and method may be implemented in a single computer environment or in a parallelized environment with multiple PC's/Servers performing varying tasks. This parallel environment could be located at just one physical space or it may be distributed at multiple remote locations connected via a computing media including but not limited to system bus, processing unit, connector cables etc.

In an example, a predictive model for dental care is integrated in a model software application for use by a dental providers. The computerized system and method is helpful in identifying dental risk for an individual patient over a period of time (e.g., one year). Historical patient specific data, including clinical, medical, and pharmacy claims data and consumer data such as demographic data, geographic data and financial data 100, is preprocessed and transformed using various well-known techniques 102, 104 before input to a predictive model 106. The preprocessing algorithms include variable selections, principle component analysis, and clustering and so on. A dental intervention predictive model 108 is developed using a combination of various well-known techniques such as those listed in FIG. 1D.

FIG. 1E is a diagram of exemplar triggers or risks factors considered relevant to the predictive model. Tooth location 172 is a first component. Decay most often occurs in back teeth (molars and premolars). The molars and premolars have grooves, pits and multiple roots that can collect food particles. The rear teeth are often harder to keep clean than the smoother, easier to reach front teeth. Another component for consideration is diet 174. Foods that cling to teeth for a long time are more likely to cause decay than foods that are easily washed away by saliva. Milk, ice cream, honey, sugar, soda, dried fruit, cake, cookies, hard candy and mints, dry cereal, and chips are examples of food that could cling to teeth. Frequent snacking 176 or sipping is another risk to consider. Steadily snacking or sipping sugary drinks, you give mouth bacteria more fuel to produce acids that attack your teeth and wear them down. And sipping soda or other acidic drinks throughout the day helps create a continual acid bath over your teeth. Another factor to consider is brushing 178. The amount of time between eating and drinking and brushing impacts plaque formation and the first stages of decay can begin. Existing fillings or devices 180, or dental history, can also be used in the model. Additionally, the condition of dental fillings and appliances can be used in the model. Over time, dental fillings can weaken, begin to break down or develop rough edges. Change in the filling condition can allow plaque to build up more easily and makes plaque harder to remove. Dental devices can also stop fitting well, allowing decay to begin underneath the device. The amount of fluoride 182 can also be relevant. Fluoride, a naturally occurring mineral, can help prevent cavities and can reverse the earliest stages of tooth damage. Age 184 can also be used in the model. For example, the United States, cavities are common in very young children and teenagers. Older adults also are at higher risk. Over time, teeth can wear down and gums may recede, making teeth more vulnerable to root decay. Older adults also may use more medications that reduce saliva flow, increasing the risk of tooth decay. Whether the person has an eating disorder 186 can also be used in the model. For example, anorexia and bulimia can lead to significant tooth erosion and cavities. Stomach acid from repeated vomiting (purging) washes over the teeth and begins dissolving the enamel. Eating disorders also can interfere with saliva production. Additionally, heartburn or gastroesophageal reflux disease (GERD) can cause stomach acid to flow into the mouth (reflux) which results in wearing away tooth enamel which can cause significant tooth damage. Exposing more of the dentin to attack by bacteria can also cause tooth decay. Dry mouth 188 can also be used in the model. Dry mouth is caused by a lack of saliva. Saliva helps prevent tooth decay by washing away food and plaque from your teeth. Substances found in saliva also help counter the acid produced by bacteria. Certain medications, some medical conditions, radiation to your head or neck, or certain chemotherapy drugs can increase your risk of cavities by reducing saliva production.

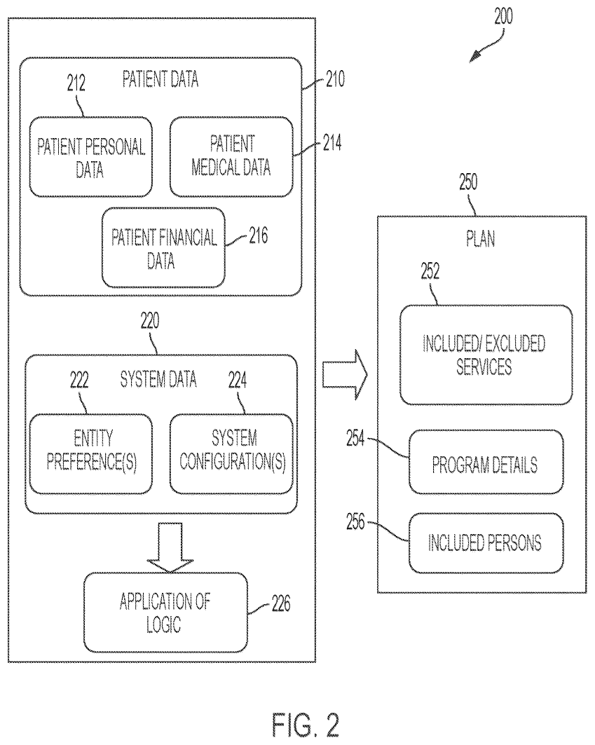

FIG. 2 illustrates a block diagram for the dental predictor system 200. Patient data 210 includes patient personal data 212, patient medical data 214, and patient financial data 216. System data 220 includes entity preferences 222, and system configurations 224. The system data 220 can also include one or more fixed or personalized settings and configurations. Fixed system data can be changed directly in the server. System configurations 224 may or may not include medical and/or non-medical data, historical and/or non-historical data, analytical and/or non-analytical data, and/or system generated and/or imported data. Application logic 226 is also provided. Patient data 210 and system data 220 can be analyzed and processed using application of logic 226. Information is then provided to the plan 250. The plan 250 includes included/excluded services 252, program details 254, and included persons 256. The plan may have a duration, may or may not include one or more types of services, may or may not include one or more types of discount for any part of the services or all the services in the program, may or may not cover a plurality of persons (e.g., family plan), may or may not include prior payment data, may cover whole or partial service costs, and may or may not include free or discounted visits. A payment mechanism can be provided that allows integration with third party systems.

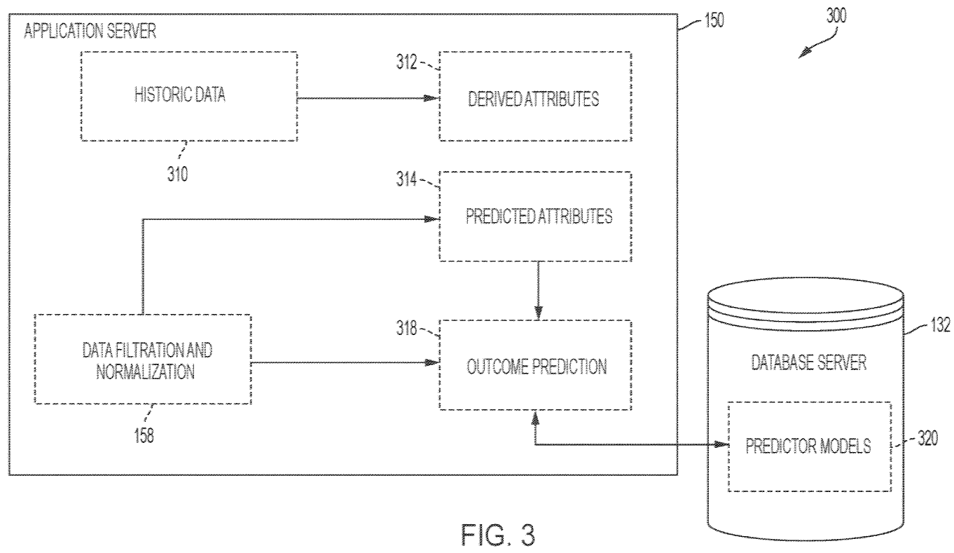

FIG. 3 is a block diagram that illustrates an exemplary scenario 300 for predicting dental outcomes and costs, in accordance with an exemplary embodiment of the disclosure. The exemplary scenario 300 involves the target patient who may provide historical data 310, the application server 130, and the database server 132 that may store the predictor models 320. The exemplary scenario 300 illustrates a scenario where the historical data 310 includes historical data 310 of the target patient, and answers provided by the target patient to any questions.

Historical data 310 of the target patient may include, but is not limited to, historical dental information, historical dental information, type of toothbrush (e.g., electric), times of brushing, educational level, travel history, employment history, etc. for the target patient. The application server 130 may be further configured to communicate a questionnaire to the target patient. Additionally, historical data 310 of the target patient can be retrieved through the software application accessed by the target patient or via the user devices 120. The user devices 120 may be configured to communicate to the application server 130 a response provided by the target patient to the questionnaire and the application server 130 may be configured to the include the response of the target patient in the historical data 310.

After retrieving historical data 310, the application server 130 may be configured to process the historical data 310. Processing of the historical data 310 may involve filtering and normalizing the historical data 310. After the historical data 310 is processed, the prediction module 156 may be configured to query the database server 132 to retrieve the predictor models 320. The prediction module 156 may be configured to use feature values extracted the analyzed historical data as input to the predictor models, respectively, for outcome prediction (as represented by block 318). The outcome prediction may yield predicted attributes 314 of the target patient as output. In one embodiment, the prediction module 156 may be configured to predict patient outcome by using the predictor model. After the outcome is predicted, the prediction module 156 may be configured to normalize and adjust the personality and mood attributes to yield the predicted attributes 314. In another embodiment, the prediction module 156 may be configured to normalize and combine the feature values extracted from the dental, dental, and bibliographic data and use the normalized and combined feature values as input to the first predictor model for obtaining the predicted attributes 314. In another example, the prediction module 156 may be configured to predict the predicted attributes 314 by using the first predictor model in two stages.

The application server 130 may be configured to store the predicted dental outcomes in the database server 132. In an embodiment, the dental outcomes may include, but are not limited to, monthly and/or annual costs, proactive preventive measures (e.g., cleaning three times per year instead of twice per year). The application server 130 may be configured to communicate the predicted dental outcomes to, or about, the target patient. Thus, based on the predicted dental outcomes, informed cost estimates over time may be made or projected by the system. The application server 130 may communicate the predicted dental outcomes to an organization. Personalization of a patient (e.g., analyzing dental, dental and bibliographic data of the patient) to understand more complex patterns of patient behavior and projected dental outcomes.

The system is operable to, for example, appraise an in-house dental plan for patients. The system receives multiple variables including but not limited to location, patient's age, number of existing decayed teeth, number of missing teeth, number of filled teeth and number of systemic diseases in order to provide a personalized plan for a patient. The weighting of the evaluated parameters can be adjusted during the evaluation process or analysis formula at any time. The plan also contains a list of treatments fully or partially covered by the dentist and the annual or monthly fee. Once the analysis is completed, one or more plans can be shown to the patient through an electronic device terminal.

In one embodiment, the application server 130 may be configured to render a user interface (UI) on the user devices 120 for presenting the predicted dental outcomes to the target patient. In one example, the application server 130 may render the UI through the software application that runs on the user devices 120. The model generator 154 may be configured to adjust the weight of links between the patient data and historic model.

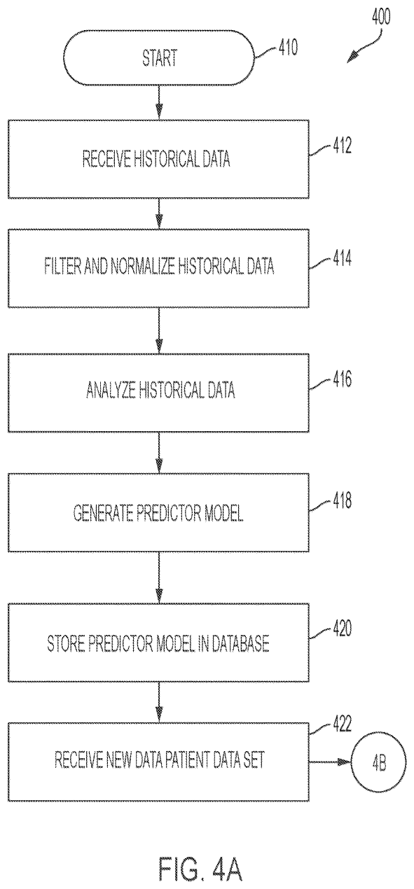

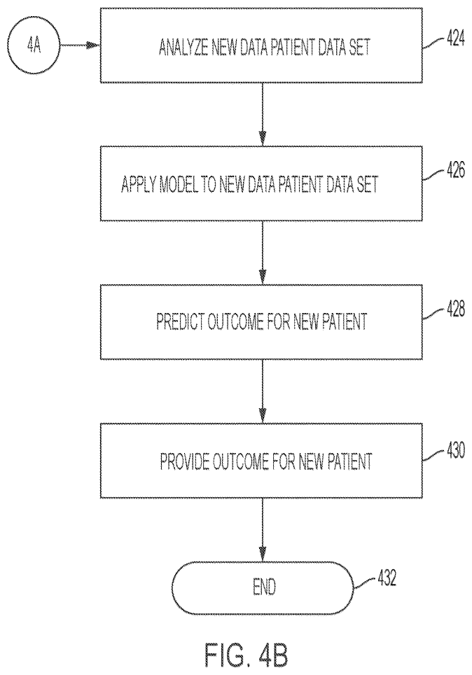

FIGS. 4A-4B, collectively represent a flow diagram 400 illustrating a method for predicting dental outcomes and costs, in accordance with an embodiment of the disclosure. At 410, the historical data 310 of a plurality of users is retrieved. The application server 130 may retrieve the historical data 310. At 412, the historical data 310 is filtered and normalized. At 414, the historical data is analyzed. At 416, the predictor models 320 for prediction of outcomes are generated. The application server 130 generates the predictor models 320 based on the historical data. At 420, the predictor models 320 are stored in the database server 132. At 422, the data is received from a target patient. At 424, the data the target patient data is analyzer. At 426, the model is applied to the target patient data set. At 428, a predicted outcome for the target patient is generated. At 430, the predicted outcome is provided. Thereafter the process ends 432.

### EXAMPLES

Analyzed data can be organized into, for example, four predictive risk categories: (1) low risk, (2) medium risk, (3) moderate risk, and (4) high risk. Within each of the four predictive risk categories, two or more variables can be analyzed and weighted from 1% to 99%, and fractions thereof. Multiple variables are selected from, for example, (a) dental office and/or patient residence (e.g., city or zip code), (b) patient medical history, (c) patient dental history, (d) patient current medical and dental condition, and (c) patient age. Other variables can be used without departing from the scope of the disclosure including, for example, presence of missing teeth. Moreover, each of the variables can be further broken into sub-variables. For example, medical history can include variables for high blood pressure, diabetes, cancer, etc.

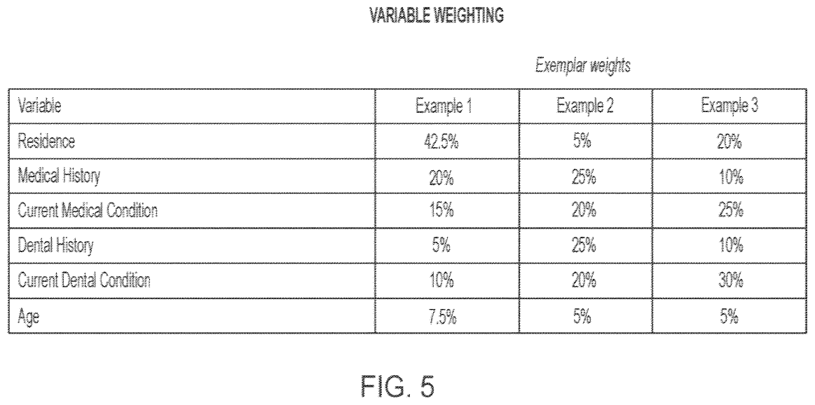

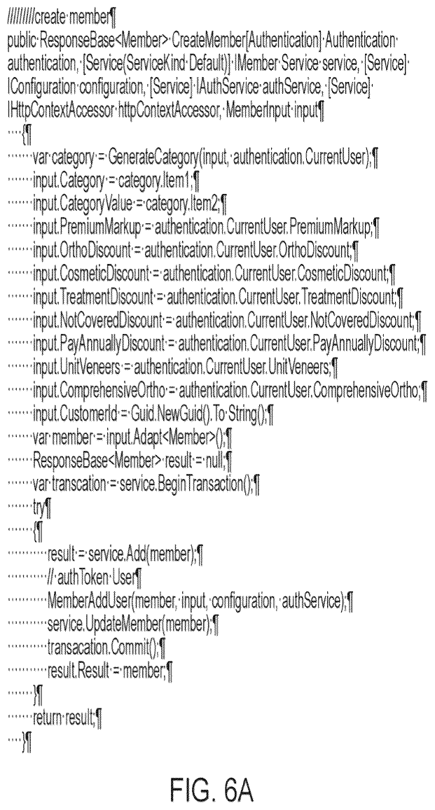

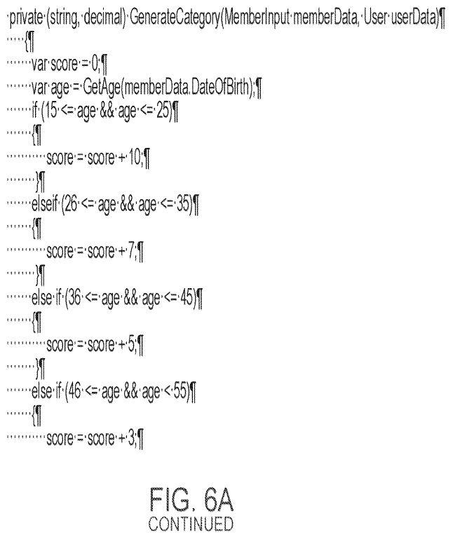

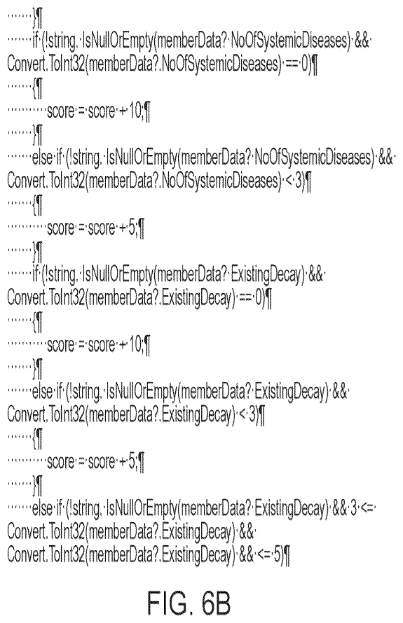

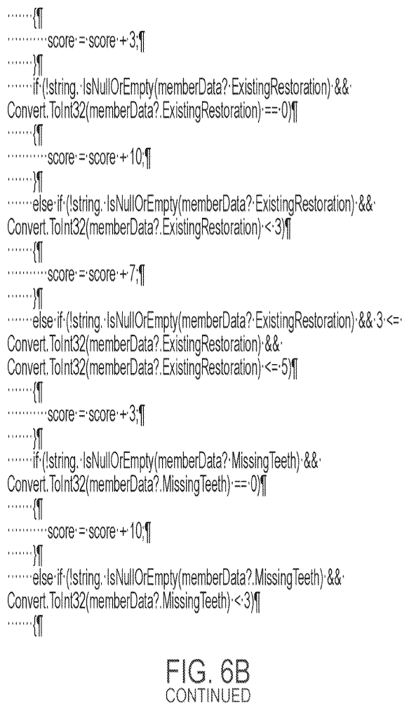

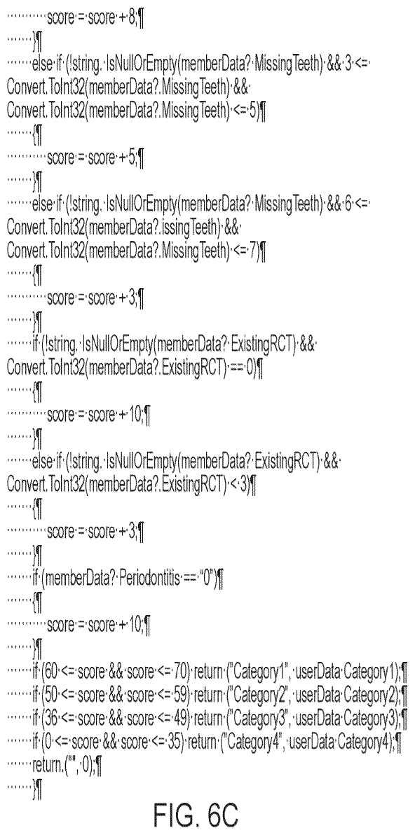

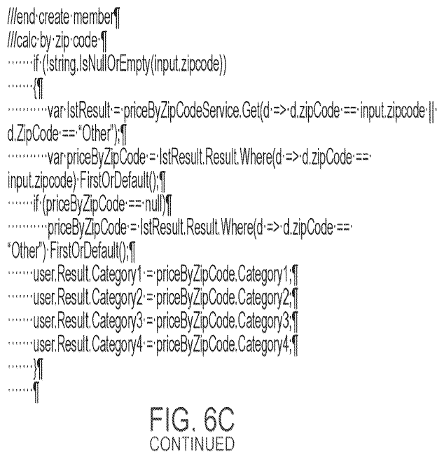

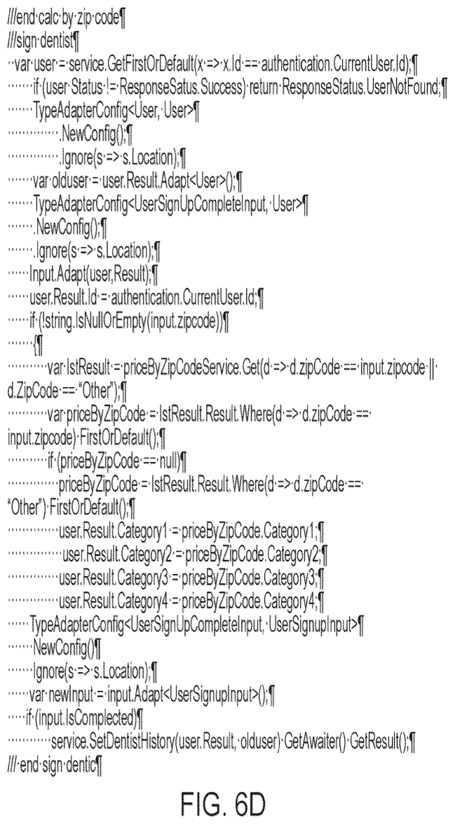

The system is configurable to process a weight to a variable as shown in FIG. 5 and use that weighted information to assign a patient to one of, for example, four risk categories. Changes in a variable or weight of a variable can result in a different outcome and a different prediction. FIGS. 6A-D illustrate exemplar software code.

### Patient 1

A 73 year old male with high blood pressure and diabetes, one missing tooth, two existing fillings and one existing root canal, with current diagnosis of one needed crown, residing in Los Angeles, California, has been assigned a risk category of high risk.

### Patient 2

A 21 year old female, with no systemic disease, no missing teeth, with one existing filling, with current diagnosis of one filling needed, residing in San Francisco, California, has been assigned a risk category of low risk.

### Patient 3

A 42 year old female, with current diagnosis of periodontal disease and two extractions needed, no existing fillings and missing two teeth, residing in Riverside, California, has been assigned a risk category of moderate risk.

### Patient 4

A 30 years old male, with no dental history, a smoker with no recorded medical issues, residing in Eureka, California, has been assigned a risk category of high risk.

### Patient 5

A 50 year old female, with gaps in her dental history, no medical issues, residing in Modesto, California, has been assigned a risk category of medium-high risk.

### Patient 6

A 73 year old male with high blood pressure and diabetes, one missing tooth, two existing fillings and one existing root canal, with current diagnosis of one needed crown, residing in Los Angeles, California, has a determined risk of high.

### Patient 7

A 21 year old female, with no systemic disease, no missing teeth, with one existing filling, with current diagnosis of one filling needed, residing in San Francisco, California, has a determined risk of low.

### Patient 8

A 42 year old female, with current diagnosis of periodontal disease and two extractions needed, no existing fillings and missing two teeth, residing in Riverside, California, has a determined risk of moderate.

### Patient 9

A 30 years old male, with no dental history, a smoker with no recorded medical issues, residing in Eureka, California, has a determined risk of high.

### Patient 10

A 50 year old female, with gaps in her dental history, no medical issues, residing in Modesto, California, has a determined risk of medium-high.

### Patient 11

A 73 year old male with high blood pressure and diabetes, one missing tooth, two existing fillings and one existing root canal, with current diagnosis of one needed crown, residing in Los Angeles, California, has been assigned to a prevention program that includes, for example, three cleanings per year and the use of an electric toothbrush.

### Patient 12

A 21 year old female, with no systemic disease, no missing teeth, with one existing filling, with current diagnosis of one filling needed, residing in San Francisco, California, has been assigned to a prevention program that includes, for example, two cleanings per year.

### Patient 13

A 42 year old female, with current diagnosis of periodontal discase and two extractions needed, no existing fillings and missing two teeth, residing in Riverside, California, has been assigned to a prevention program that includes, for example, three cleanings per year, flossing twice per day, and the use of an electric toothbrush.

### Patient 14

A 30 years old male, with no dental history, a smoker with no recorded medical issues, residing in Eureka, California, has been assigned to a prevention program that includes, for example, three cleanings per year and the use of an electric toothbrush.

### Patient 15

A 50 year old female, with gaps in her dental history, no medical issues, residing in Modesto, California, has been assigned to a prevention program that includes, for example, three cleanings per year and the use of an electric toothbrush.

It will be understood by a person of ordinary skill in the art that the abovementioned dental outcomes are listed for exemplary purpose and should not be construed to limit the scope of the disclosure. In other embodiments, the predictor models 320 may be utilized to predict dental outcomes that are different from the dental outcomes mentioned above.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that any claims presented define the scope of the invention and that methods and structures within the scope of these claims and their equivalents be covered thereby.

1. A computer-implemented system for assigning a dental patient to a risk category, the system comprising:
one or more computing devices storing
a dental risk model;
dental intervention predictors comprising one or more of tooth location, diet, snacking pattern, brushing habits, existing fillings, existing devices, fluoride use, age, eating disorders, dry mouth, heartburn, and acid reflux; and
one or more computing devices executing instructions to
receive patient specific data for a patient and a population dataset for a patient population, wherein the patient specific data comprises data selected from the group comprising medical claims data and pharmacy claims data, wherein population data comprises data selected from the group comprising demographic data, geographic data, and financial data;
analyze the population data to identify a subset of the population dataset having one or more of the dental risk model triggers present in the patient specific data;
process the patient specific data and the subset of the population dataset using an algorithm selected from the group comprising variable selection, principle component analysis, and clustering;
extract features from the patient specific data by temporal feature extraction;,
generate a predicted dental intervention;
provide a plurality of training conditions to a computing device wherein the training conditions comprise tooth location, diet, snacking pattern, brushing habits, existing fillings, existing devices, fluoride use, age, eating disorders, dry mouth, heartburn, and acid reflux;
develop the dental intervention predictive model using a modeling technique selected from the group comprising decision tree, logistic regression, artificial neural networks, and ensemble;
provide the patient specific data and the population dataset to the computing device that comprises the dental intervention predictive model;
receive a calculated risk score from the computing device that comprises the dental intervention predictive model, wherein the dental risk score represents the likelihood that the patient will require a dental procedure within a predetermined time period, and wherein the risk score is determined at least in part based on the presence or absence of each of the dental predictors in the patient specific data for the patient;
sort the received calculated risk score into one of a plurality of groups according to a severity of risk determined by the calculated risk score; and
assign a program or intervention for the patient, wherein the assignment is determined based on the calculated risk score.

2. The computer-implemented system of claim 1 further comprising the operation of:
enroll the patient in the assigned program or intervention.

3. The computer-implemented system of claim 1 wherein the intervention is adapted to reduce an actual intervention from the calculated intervention.

4. The computer-implemented system of claim 1 wherein one or more data components in the population dataset is weighted.

5. The computer-implemented system of claim 1 further comprising the operation of:
revise a weight of a data component of the population dataset after the calculated risk score is received.

6. A method comprising:
providing a computer-implemented system for assigning a dental patient to a risk category, the system comprising:
one or more computing devices storing
a dental risk model;
dental intervention predictors comprising one or more of tooth location, diet, snacking pattern, brushing habits, existing fillings, existing devices, fluoride use, age, eating disorders, dry mouth, heartburn, and acid reflux; and
one or more computing devices executing instructions to
receive patient specific data for a patient and a population dataset for a patient population, wherein the patient specific data comprises data selected from the group comprising medical claims data and pharmacy claims data, wherein population data comprises data selected from the group comprising demographic data, geographic data, and financial data;
analyze the population data to identify a subset of the population dataset having one or more of the dental risk model triggers present in the patient specific data;
process the patient specific data and the subset of the population dataset using an algorithm selected from the group comprising variable selection, principle component analysis, and clustering;
extract features from the patient specific data by temporal feature extraction;
generate a predicted dental intervention;
provide a plurality of training conditions to a computing device wherein the training conditions comprise tooth location, diet, snacking pattern, brushing habits, existing fillings, existing devices, fluoride use, age, eating disorders, dry mouth, heartburn, and acid reflux;
develop the dental intervention predictive model using a modeling technique selected from the group comprising decision tree, logistic regression, artificial neural networks, and ensemble;
provide the patient specific data and the population dataset to the computing device that comprises the dental intervention predictive model;
receive a calculated risk score from the computing device that comprises the dental intervention predictive model, wherein the dental risk score represents the likelihood that the patient will require a dental procedure within a predetermined time period, and wherein the risk score is determined at least in part based on the presence or absence of each of the dental predictors in the patient specific data for the patient;
sort the received calculated risk score into one of a plurality of groups according to a severity of risk determined by the calculated risk score; and
assign a program or intervention for the patient, wherein the assignment is determined based on the calculated risk score.

7. The method of claim 6 further comprising the operation of:
enroll the patient in the assigned program or intervention.

8. The method of claim 6 wherein the intervention is adapted to reduce an actual intervention from the calculated intervention.

9. The method of claim 6 wherein the one or more data components in the population dataset is weighted.

10. The method of claim 6 further comprising the operation of:
revise a weight of a data component of the population dataset after the calculated risk score is received.

11. A computer-implemented system for determining a dental risk for a dental patient, the system comprising:
one or more computing devices storing
a dental risk model;
dental intervention predictors comprising one or more of tooth location, diet, snacking pattern, brushing habits, existing fillings, existing devices, fluoride use, age, eating disorders, dry mouth, heartburn, and acid reflux; and
one or more computing devices executing instructions to
receive patient specific data for a patient and a population dataset for a patient population, wherein the patient specific data comprises data selected from the group comprising medical claims data and pharmacy claims data, wherein population data comprises data selected from the group comprising demographic data, geographic data, and financial data;
analyze the population data to identify a subset of the population dataset having one or more of the dental risk model triggers present in the patient specific data; process the patient specific data and the subset of the population dataset using an algorithm selected from the group comprising variable selection, principle component analysis, and clustering;
extract features from the patient specific data by temporal feature extraction;
generate a predicted dental intervention;
provide a plurality of training conditions to a computing device wherein the training conditions comprise tooth location, diet, snacking pattern, brushing habits, existing fillings, existing devices, fluoride use, age, eating disorders, dry mouth, heartburn, and acid reflux;
develop the dental intervention predictive model using a modeling technique selected from the group comprising decision tree, logistic regression, artificial neural networks, and ensemble;
provide the patient specific data and the population dataset to the computing device that comprises the dental intervention predictive model;
receive a calculated risk score from the computing device that comprises the dental intervention predictive model, wherein the dental risk score represents the likelihood that the patient will require a dental procedure within a predetermined time period, and wherein the risk score is determined at least in part based on the presence or absence of each of the dental predictors in the patient specific data for the patient; and
determine the dental risk for the dental patient.

12. The computer-implemented system of claim 11 further comprising the operations of:
assign a program or intervention for the patient, wherein the assignment is determined based on the calculated risk score; and
enroll the patient in the assigned program or intervention.

13. The computer-implemented system of claim 12 wherein the intervention is adapted to reduce an actual intervention from the calculated intervention.

14. The computer-implemented system of claim 11 wherein one or more data components in the population dataset is weighted.

15. The computer-implemented system of claim 11 further comprising the operation of:
revise a weight of a data component of the population dataset after the calculated risk score is received.

16. A computer-implemented system for assigning a prevention program based on a dental risk for a dental patient, the system comprising:
one or more computing devices storing
a dental risk model;
dental intervention predictors comprising one or more of tooth location, diet, snacking pattern, brushing habits, existing fillings, existing devices, fluoride use, age, eating disorders, dry mouth, heartburn, and acid reflux; and
one or more computing devices executing instructions to
receive patient specific data for a patient and a population dataset for a patient population, wherein the patient specific data comprises data selected from the group comprising medical claims data and pharmacy claims data, wherein population data comprises data selected from the group comprising demographic data, geographic data, and financial data;
analyze the population data to identify a subset of the population dataset having one or more of the dental risk model triggers present in the patient specific data;
process the patient specific data and the subset of the population dataset using an algorithm selected from the group comprising variable selection, principle component analysis, and clustering;
extract features from the patient specific data by temporal feature extraction;
generate a predicted dental intervention;
provide a plurality of training conditions to a computing device wherein the training conditions comprise tooth location, diet, snacking pattern, brushing habits, existing fillings, existing devices, fluoride use, age, eating disorders, dry mouth, heartburn, and acid reflux;
develop the dental intervention predictive model using a modeling technique selected from the group comprising decision tree, logistic regression, artificial neural networks, and ensemble;
provide the patient specific data and the population dataset to the computing device that comprises the dental intervention predictive model;
receive a calculated risk score from the computing device that comprises the dental intervention predictive model, wherein the dental risk score represents the likelihood that the patient will require a dental procedure within a predetermined time period, and wherein the risk score is determined at least in part based on the presence or absence of each of the dental predictors in the patient specific data for the patient; and
assign a program or intervention for the patient, wherein the assignment is determined based on the calculated risk score.

17. The computer-implemented system of claim 16 further comprising the operation of:
enroll the patient in the assigned program or intervention.

18. The computer-implemented system of claim 16 wherein the intervention is adapted to reduce an actual intervention from the calculated intervention.

19. The computer-implemented system of claim 16 wherein one or more data components in the population dataset is weighted.

20. The computer-implemented system of claim 16 further comprising the operation of:
revise a weight of a data component of the population dataset after the calculated risk score is received.
